(19) United States
(12) Patent Application Publication
(10) Pub. No.: US 2025/0114500 A1
(43) Pub. Date: Apr. 10, 2025

(54) FLEXIBLE MEMBRANE MAGNETICALLY DRIVEN TO GENERATE NEGATIVE PRESSURE, AND BREAST PUMP

(71) Applicant: Shenzhen TPH Technology Co.,Ltd., Shenzhen (CN)

(72) Inventor: Xianzi CHEN, Shenzhen (CN)

(21) Appl. No.: 18/985,424

(22) Filed: Dec. 18, 2024

Related U.S. Application Data
(63) Continuation of application No. PCT/CN2024/123678, filed on Oct. 9, 2024.

(30) Foreign Application Priority Data
Sep. 29, 2024 (CN) 202422388121.X

(51) Int. Cl. A61M 1/06

(52) U.S. Cl. CPC A61M 1/06; A61M 2205/106; A61M 2205/42

(57) ABSTRACT

A flexible membrane magnetically driven to generate negative pressure includes a mounting portion, a deformation portion and a magnetic part embedded within the deformation portion. The mounting portion is configured for mounting the flexible membrane. The deformation portion is integrally formed with the mounting portion. A connection between the deformation portion and the mounting portion is sealed. The mounting portion is provided at an edge of the deformation portion. The magnetic part is configured to magnetically drive the formation of the deformation portion.

### CROSS-REFERENCE TO RELATED APPLICATIONS

This application is a continuation of International Patent Application No. PCT/CN2024/123678, filed on Oct. 9, 2024, which claims the benefit of priority from Chinese Patent Application No. 202422388121.X, filed on Sep. 29, 2024. The content of the aforementioned application, including any intervening amendments thereto, is incorporated herein by reference in its entirety.

### TECHNICAL FIELD

This application relates to breast pumps, and more particularly to a flexible membrane magnetically driven to generate negative pressure, and a breast pump.

### BACKGROUND

A breast pump is designed to express the breast milk from the mammary glands. The breast pump is applicable when a baby is unable to directly suckle at the breast or when there is an excess of breast milk.

In the existing breast pumps, vacuum pumping and mechanical drive are often adopted to deform the flexible membranes. However, these methods are generally accompanied by the generation of strong noise, which may adversely affect the user's emotion. Therefore, there is an urgent need to develop a flexible membrane that can be driven by an alternative approach to deform, thereby addressing the issue of excessive noise.

### SUMMARY

In view of this, in a first aspect, this application provides a flexible membrane magnetically driven to generate negative pressure, comprising:
a mounting portion;
a deformation portion; and
a first magnetic part;
wherein the mounting portion is configured for mounting the flexible membrane;
the deformation portion is integrally formed with the mounting portion, a connection between the deformation portion and the mounting portion is sealed, and the mounting portion is provided at an edge of the deformation portion; and
the first magnetic part is embeddedly provided within the deformation portion.

In a second aspect, this application provides a magnetically-driven breast pump, comprising:
the above flexible membrane.

Compared to the prior art, the present disclosure has the following beneficial effects.

By embedding the first magnetic part, the deformation portion can be magnetically driven to be deformed, and thus there is no need for a vacuum pump or mechanical drive. Therefore, the flexible membrane designed herein can effectively prevent the noise generation, and will not affect the user's emotion.

### BRIEF DESCRIPTION OF THE DRAWINGS

In order to illustrate the technical solutions in the embodiments of the present disclosure or the prior art more clearly, the accompanying drawings needed in the description of the embodiments or prior art will be briefly described below. Obviously, presented in the accompanying drawings are only some embodiments of the present disclosure, and for those of ordinary skill in the art, other accompanying drawings can be obtained from the structures illustrated therein without making creative effort.

FIG. 1 schematically shows an overall structure of a breast pump magnetically driven to generate negative pressure according to an embodiment of the present disclosure;

FIG. 2 is an exploded view of the breast pump according to an embodiment of the present disclosure;

FIG. 3 schematically shows an assembly structure of the breast pump according to an embodiment of the present disclosure;

FIG. 4 is a sectional view of the breast pump according to an embodiment of the present disclosure;

FIG. 5 is an enlarged view of portion “A” in FIG. 4;

FIG. 6 schematically shows a structure of a connecting part of the breast pump according to an embodiment of the present disclosure;

FIG. 7 schematically shows a structure of a cover of the breast pump according to an embodiment of the present disclosure;

FIG. 8 schematically shows a structure of a flexible membrane of the breast pump according to an embodiment of the present disclosure;

FIG. 9 schematically shows an internal structure of the flexible membrane according to an embodiment of the present disclosure;

FIG. 10 is a structural diagram of a control system for a wearable breast pump according to an embodiment of the present disclosure; and

FIG. 11 is a flow chart of a control method for the wearable breast pump according to an embodiment of the present disclosure.

In the figures: 1—first housing; 11—breast shield; 111—first cavity; 112—second cavity; 12—cover; 121—first mounting portion; 122—convex ring; 123—assembly slot; 124—through-hole; 125—air groove; 126—first quick-release part; 127—liquid outlet; 2—second housing; 21—first half shell; 211—button; 212—charging port; 22—second half shell; 221—second quick-release part; 3—connecting part; 31—mounting cylinder; 311—limiting part; 32—first connecting tube; 33—second connecting tube; 34—mounting structure; 341—first clamping portion; 4—flexible part; 4a—second mounting portion; 4b—deformation portion; 41—first magnetic part; 42—second clamping portion; 43—annular groove; 44—protrusion; 45—third magnetic part; 46—trough; 47—air hole; 5—second magnetic part; 6—control board; 7—power supply device; and 8—check valve.

The implementation, functional characteristics, and advantages of the present disclosure will be further described in conjunction with the embodiments and the accompanying drawings.

### DETAILED DESCRIPTION OF EMBODIMENTS

The technical solutions of the present disclosure will be described clearly and completely below in conjunction with the accompanying drawings. It is obvious that described herein are merely some embodiments of the present disclosure, rather than all embodiments of the present disclosure. Based on the embodiments in the present disclosure, all other embodiments obtained by those of ordinary skill in the art without making creative effort shall fall within the scope of the present disclosure defined by the appended claims.

It should be noted that all directional indications (such as up, down, left, right, front, and back) in the embodiments of the present disclosure are used only for explaining the relative positional relationship or movement between the components in a particular attitude (as shown in the accompanying drawings), and the directional indications are correspondingly changed if the particular attitude is changed.

Furthermore, as used herein, terms such as “first” and “second” are only descriptive, and should not be construed as indicating or implying their relative importance or implicitly specifying the number of technical features indicated. As a result, a feature defined as “first” or “second” may include at least one of such feature, either explicitly or implicitly. In addition, “and/or” includes three solutions, for example, “A and/or B” includes technical solution A, technical solution B, and a combination thereof. In addition, the technical solutions of various embodiments may be combined with each other on the premise that the combined solution can be implemented by those of ordinary skill in the art. When the combination of technical solutions appears to be contradictory or unimplementable, it should be understood that such a combination does not exist, and is not included within the scope of the present disclosure.

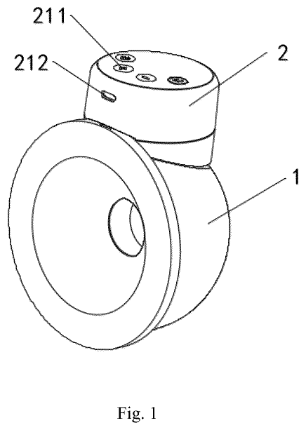

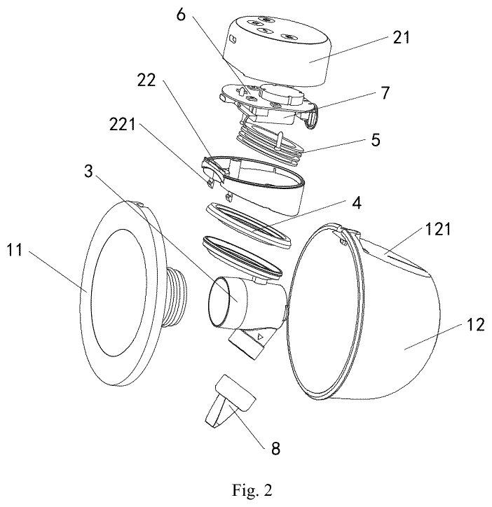

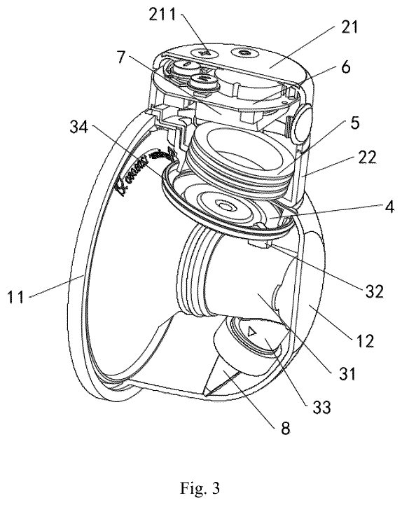

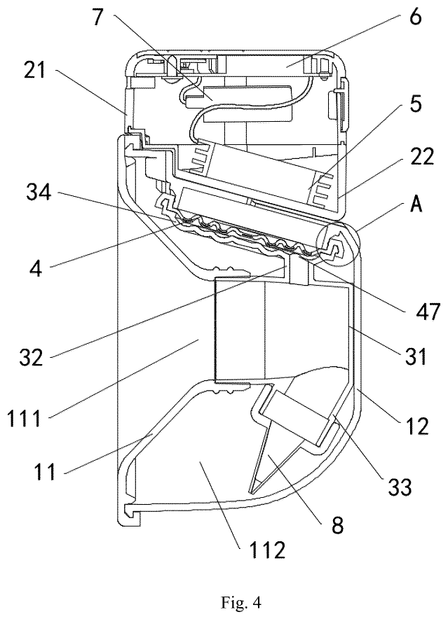

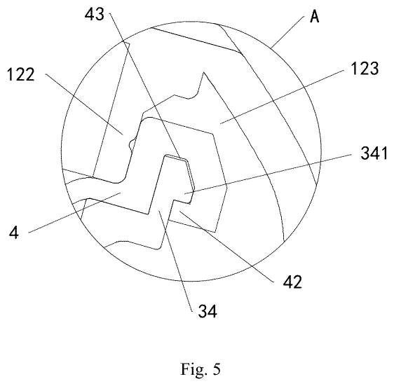

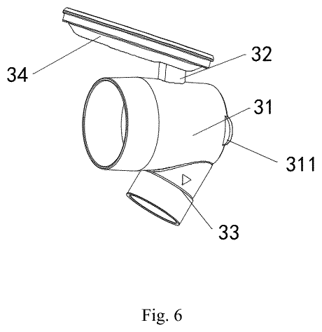

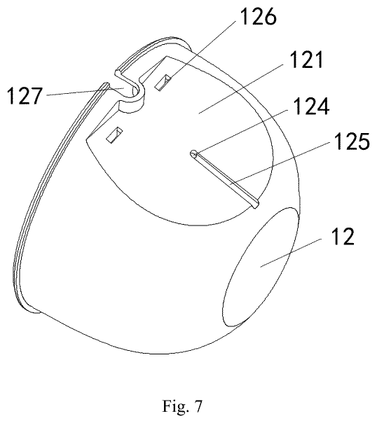

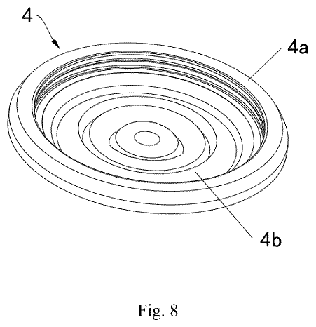

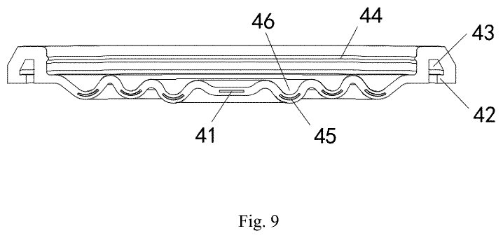

As shown in FIGS. 1-9, an embodiment of the present application provides a breast pump magnetically driven to generate negative pressure, including: a breast shield 11 in a flared shape, a flexible part 4 provided with a first magnetic part 41, and a second magnetic part 5. The breast shield 11 defines a first cavity 111, which is configured to closely fit and at least partially accommodate a breast to form a sealed cavity. The flexible part 4 is at least partially communicated with the sealed cavity and forms a portion of a cavity wall of the sealed cavity. The first magnetic part 41 is provided opposite to the second magnetic part 5, and the second magnetic part 5 is configured to drive the first magnetic part 41 to move and drive the flexible part 4 to deform through magnetic force, thereby generating a negative pressure within the sealed cavity.

In this embodiment, an inner wall of the breast shield 11 is configured to fit the user's breast. When the inner wall of the breast shield 11 is attached to the user's breast, the sealed cavity enclosed by the breast shield 11 and the flexible part 4 is in a sealed state.

The first magnetic part 41 is provided opposite to the second magnetic part 5, and their positions can be interchanged. It should be noted that the term “magnetic part” used herein is not limited to those parts with magnetic properties, and materials that can be magnetically driven to move are also encompassed.

In some embodiments, the first magnetic part 41 includes but is not limited to permanent magnets. The first magnetic part 41 can be annular, block-shaped or strip-shaped. The second magnetic part 5 is an electromagnetic coil, and can be annular, block-shaped or strip-shaped.

During operation, the breast shield 11 is attached firmly to the user's breast to allow the inner wall of the breast shield 11 fit the user's breast snugly. Then, the second magnetic part 5 is activated to generate a magnetic force to drive the movement of the first magnetic part 41. Specifically, when the magnetic pole of the second magnetic part 5 is opposite to that of the first magnetic part 41, a magnetic attraction force is generated between the first magnetic part 41 and the second magnetic part 5. Under the action of the magnetic attraction force, the flexible part 4 is driven to be deformed, creating a negative pressure within the sealed cavity. At this time, the breast shield 11 will apply a pressure on the user's breast to express the breast milk. Conversely, when the magnetic pole of the second magnetic part 5 is the same as that of the first magnetic part 41, a repulsive force is generated therebetween to return the flexible part 4 to its original position, and the negative pressure within the sealed cavity disappears.

In this embodiment, a traditional breast pumping form formed by a vacuum pump and a solenoid valve is replaced with an electromagnetic breast pumping form, significantly reducing a weight of the breast pump for enhanced portability. Additionally, the elimination of the vacuum pump and the solenoid valve can effectively reduce and even eliminate noise. As a result, the breast pump designed herein can alleviate the impact of the breast milk pumping process on the mother's emotions and enhance the comfortability.

Furthermore, this application replaces components such as the vacuum pump and solenoid valve with a magnetic attraction mechanism, reducing the number of assembly components (such as the vacuum pump, solenoid valve, sealing rings, etc.) and effectively lowering costs.

In an embodiment, the flexible part 4 further includes at least one third magnetic part 45, which is provided at a periphery of the first magnetic part 41.

In this embodiment, by embedding the third magnetic part 45 within the flexible part 4, the magnetic strength can be further enhanced, thereby improving efficiency and reducing energy loss. And the third magnetic part 45 is a neodymium magnet.

The third magnetic part 45 has an annular-shape. When the number of the third magnetic parts 45 is two or more, the third magnetic parts 45 are arranged in an ascending manner in diameter from inside to outside along a radial direction of the flexible part 4.

In an embodiment, the sealed cavity is communicated with a milk collection mechanism, which is configured to collect and store breast milk.

In this embodiment, any container that can collect and store breast milk can be considered a milk collection mechanism, such as a baby bottle or a bowl-shaped container.

In an embodiment, the milk collection mechanism includes a cover 12, the cover 12 and the breast shield 11 are enclosed to form a first housing 1. A second cavity 112 is provided within the first housing 1, and a tapered end of the breast shield 11 is configured to extend into the second cavity 112.

In this embodiment, a connection between the breast shield 11 and the cover 12 is sealed. The cover 12 has a hemispherical-shaped. An edge of an opening of the cover 12 is further provided with a liquid outlet 127, which is configured to pour out the breast milk after breast pumping.

In an embodiment, the breast pump further includes a second housing 2, in which the second magnetic part 5 is fixed. The second magnetic part 5 is configured to alternately switch between a S pole and a N pole.

In this embodiment, by alternately switching the second magnetic part 5 between the S pole and the N pole, the flexible part 4 is driven to achieve a reciprocating motion of deformation and reset, effectively creating a stable “suction” and “release” function, enabling the breast pump to operate in a continuous and stable milking process.

Specifically, when the first magnetic part 41 serves as the S pole and the second magnetic part 5 is the N pole, the magnetic attraction force is generated between the first magnetic part 41 and the second magnetic part 5. Under the action of the magnetic attraction force, the flexible part 4 is driven to be deformed, creating the negative pressure within the sealed cavity, thus facilitating the milking operation. Conversely, when the magnetic pole of the second magnetic part 5 is the same S pole as that of the first magnetic part 41, the repulsive force is generated therebetween to return the flexible part 4 to its original position, and the negative pressure within the sealed cavity disappears.

The vacuum pump exhibits a delay in generating negative pressure, and improvements in efficiency are solely dependent on the rebound properties of the original materials. Air must be evacuated from the housing and flexible part 4 to create corresponding negative pressure at the user's body. In contrast, using the magnetic attraction method can instantly change the direction of the magnetic field and the magnitude of its strength. The repulsive force can quickly reset the flexible part 4, reducing delays and significantly enhancing the efficiency of milk extraction.

In an embodiment, the breast pump further includes a connecting part 3. A place between the breast shield 11 and the flexible part 4 is defined as a sealed cavity through the connecting part 3. The connecting part 3 has a hollow structure. One end of the connecting part 3 is provided with an opening. The tapered end of the breast shield 11 is connected to the opening of the connecting part 3.

In this embodiment, the connecting part 3 includes but is not limited to a three-port valve. The connections between the breast shield 11, the flexible part 4, and the connecting part 3 are all detachable connections, and these connections are sealed.

In an embodiment, the connecting part 3 is communicated with the milk collection mechanism, and a check valve 8 is provided between the connecting part 3 and the milk collection mechanism.

In this embodiment, through the check valve 8, when the negative pressure is generated within the sealed cavity, it can prevent the breast milk in the milk collection mechanism from flowing back into the sealed cavity. The check valve 8 is a duckbill valve. Specifically, when the negative pressure occurs in the sealed cavity, the air pressure inside the sealed cavity is less than the external air pressure, causing the check valve 8 to be deformed and closed. Additionally, once check valve 8 is closed, it prevents gas leakage, enhancing airtightness and improving milking efficiency. When the negative pressure disappears, the check valve 8 returns to its open state, allowing the collected breast milk to flow from the check valve 8 into the cover 12.

In an embodiment, the connecting part 3 is provided with a mounting structure 34, which is internally communicated with the connecting part 3. And the flexible part 4 is provided within the mounting structure 34.

In this embodiment, the mounting structure 34 is a suction bowl. The mounting structure 34 includes structures of various shapes, such as circular, square, or planar designs, provided they can securely hold the flexible part 4. All such structures are included within the scope of the mounting structure 34 in this application. The edges of the flexible part 4 are sealed or interference-fitted to the edges of the mounting structure 34, ensuring that the interior of the mounting structure 34 is in a relatively sealed state. In some embodiments, the shape of the flexible part 4 is configured to align with that of the mounting structure 34.

An air hole 47 is provided at a bottom of the mounting structure 34, such that the interior of the mounting structure 34 is communicated with the connecting part 3 through the air hole 47.

In an embodiment, the mounting structure 34 is attached to the flexible part 4.

In this embodiment, through the configuration of the mounting structure 34 and the flexible part 4 attaching to each other, there will be no gap therebetween. During the milking operation, if breast milk is accidentally drawn into the mounting structure 34, the rebound of the flexible part 4 will promptly expel the milk trapped within the mounting structure 34, preventing any residual milk from remaining inside.

In an embodiment, a convex ring 122 is provided on an inner wall of the cover 12, and the cover 12 is connected to the mounting structure 34 through the convex ring 122. The convex ring 122 is configured to extend from an opening of the mounting structure 34 into its interior. The convex ring 122 and the mounting structure 34 are configured to clamp the flexible part 4.

In this embodiment, a shape of the convex ring 122 fits the bowl-like shape of the mounting structure 34. An assembly slot 123 is formed by on one side of the convex ring 122 and an inner wall of the cover 12. When the flexible part 4 is correctly provided within the mounting structure 34, the entire assembled mounting structure 34 will be inserted into the assembly slot 123. At this point, the convex ring 122 can enter the interior of the mounting structure 34, completing the entire assembly process. Meanwhile, the convex ring 122 also serves a positioning function to ensure the stability of the installation.

In addition, when a distance between an outer wall of the convex ring 122 and an inner wall of the mounting structure 34 is larger than a thickness of the flexible part 4, if the flexible part 4 deforms, the convex ring 122 can enhance a strength at a connection between the flexible part 4 and the mounting structure 34, preventing the sealing performance from being reduced caused by the deformation of the flexible part 4. When the distance between the outer wall of the convex ring 122 and the inner wall of the mounting structure 34 is slightly less than the thickness of the flexible part 4, the strength at the connection between the flexible part 4 and the mounting structure 34 can be increased. Additionally, the convex ring 122 and the mounting structure 34 can clamp the flexible part 4, forming an interference fit that ensures a close contact between the flexible part 4 and the inner wall of the mounting structure 34, further enhancing the sealing effect.

In an embodiment, the cover 12 is provided with a first mounting portion 121, which is configured as a slot or magnetic attachment structure. The cover 12 is detachably connected to the second housing 2 through the first mounting portion 121.

In an embodiment, a through-hole 124 is provided on a surface of the cover 12, which is communicated with a cavity formed by the cover 12 and the flexible part 4.

In this embodiment, via the through-hole 124, the air pressure in the cavity formed by the cover 12 and the flexible part 4 is consistent with atmospheric pressure, enabling the flexible part 4 to return to its original position.

In an embodiment, an air groove 125 is provided on the surface of the cover 12, and the gas groove 125 is communicated with the through-hole 124.

In this embodiment, when the through-hole 124 is provided on the first mounting portion 121, an interior of the cover 12 can still communicate with the outside through the air groove 125, ensuring that the air pressure in the cavity formed by the cover 12 and the flexible part 4 remains consistent with atmospheric pressure, thereby remaining unaffected by the second housing 2.

In an embodiment, a control mechanism provided within the second housing 2 is configured to control the second magnetic part 5.

In this embodiment, the control mechanism is configured to control the magnetic poles of the second magnetic part 5, allowing it to continuously alternate between the S pole and the N pole.

In an embodiment, the control mechanism includes a control board 6 and a power supply device 7. The control board 6 is electrically connected to the second magnetic part 5, and the power supply device 7 is electrically connected to the control board 6.

In this embodiment, the power supply device 7 is a battery, with a quantity of one. In some embodiments, the number of the batteries can be two or more. The power supply device 7 is configured to supply power to the second magnetic part 5 and the control board 6.

Specifically, when power is supplied to the second magnetic component 5, the current will cause the second magnetic part 5 to generate a magnetic field, thereby producing magnetic poles. The control board 6 is configured to control the direction and magnitude of the current, thus controlling the magnetic pole conversion of the second magnetic part 5 as well as the strength of its magnetic field.

More specifically, when the first magnetic part 41 is configured as the S pole, and the current direction causes the second magnetic part 5 to become the N pole, the magnetic attraction force occurs between the first magnetic part 41 and the second magnetic part 5, creating the negative pressure within the sealed cavity to facilitate the milking operation. Conversely, when the current direction causes the second magnetic part 5 to have the S pole, the repulsion force is generated between the first magnetic part 41 and the second magnetic part 5, pushing the flexible part 4 away from the second housing 2 and causing the negative pressure within the cavity to disappear.

In this process, by precisely controlling the magnitude of the current, both the strength of the magnetic field and the level of suction can be accurately controlled, ultimately enabling the regulation of negative pressure and effectively enhancing comfort.

In an embodiment, the second housing 2 includes a first half shell 21 and a second half shell 22, and the first half shell 21 is detachably connected to the second half shell 22. A lower end of the second half shell 22 is provided with a second quick-release part 221, and is detachably provided on the first mounting portion 121 through the second quick-release part 221.

In this embodiment, the design features a detachable connection between the first half shell 21 and the second half shell 22, along with a detachable installation of the lower end of the second half shell 22 at the first mounting portion 121, which facilitates efficient assembly and disassembly of the breast pump.

The first mounting portion 121 is provided with a first quick-release part 126 corresponding to the second quick-release part 221. When the first quick-release part 126 is a snap-fit hole, the second quick-release part 221 can be a buckle. Additionally, both the first quick-release part 126 and the second quick-release part 221 can be magnetic parts.

In an embodiment, the first half shell 21 is provided with a plurality of buttons 211.

In this embodiment, the plurality of buttons 211 are configured to send various control commands to the controller, enabling different functions.

Specifically, the buttons 211 include a power button, a level button, a pause button, and a mode switch button. The power button is configured to control the starting and stopping of the breast pump. The level button consists of a “+” button to increase the level and a “−” button to decrease it. The pause button is configured to temporarily suspend the operation of the breast pump. The breast pump provided herein has a massage mode, a pumping mode, a stimulation mode, and an automatic mode. The mode switch button is configured to toggle between these four modes.

In an embodiment, the cover 12 is transparent, and an outer surface of the cover 12 is provided with a plurality of graduation lines.

In this embodiment, the cover 12 is configured as transparent, allowing for constant observation of the milk collection status and internal cleanliness, facilitating timely cleaning and preventing bacterial growth.

Additionally, the outer surface of the cover 12 is provided with graduated lines and different labels, which is configured to measure the amount of expressed breast milk.

In an embodiment, the first half shell 21 is provided with a display screen, which is electrically connected to the control board 6.

In this embodiment, the display screen can show information such as pressure, battery level, charging status, time, and level settings.

In an embodiment, the connecting part 3 includes a mounting cylinder 31, a first connecting tube 32, and a second connecting tube 33. One end of the mounting cylinder 31 has an opening, and an end of the opening of the mounting cylinder 31 is detachably connected to the tapered end of the breast shield 11. A first side of the mounting cylinder 31 is provided with the air hole 47, which is communicated with the mounting structure 34 via the first connecting tube 32. And the second side of the mounting cylinder 31 is communicated with the cover 12 through the second connecting tube 33. The check valve 8 is telescopically sleeved in the second connecting tube 33, which is away from the end of the mounting cylinder 31, and the first connecting tube 32 is provided on the second connecting tube 33.

In this embodiment, the end of the opening of the mounting cylinder 31 is connected to the tapered end of the breast shield 11 using a sealed connection or an interference fit to achieve relative sealing at the connection point. The mounting cylinder 31 provided herein has a circular-shaped cross section, but in some embodiments, it can be polygonal-shaped or other irregular-shapes. Through the second connecting tube 33, the breast milk inside the mounting cylinder 31 can flow into the cover 12 for storage.

In an embodiment, the connecting part 3 is provided with a limiting part 311, and the connecting part 3 is configured to restrict relative movement between the connecting part 3 and the milk collection mechanism through the limiting part 311.

In this embodiment, the limiting part 311 is in a semi-circular plate sheet shape, with at least two such limiting parts provided on both sides of the connecting part 3. Through the limiting parts 311, the connecting part 3 can be restricted and positioned during assembly with the cover 12.

In an embodiment, an outer edge of the opening of the mounting structure 34 is provided with a first clamping portion 341. The bowl wall of the mounting structure 34 and the first clamping portion 341 are configured as a hook structure, and an upper end of the hook structure is configured to incline toward a bottom of the cover 12. An outer edge of the flexible part 4 is configured to bend downward to form an annular groove 43. An edge of the flexible part 4 is provided with a second clamping portion 42, which is configured to extend into an interior of the annular groove 43. When the first clamping portion 341 is inserted into the annular groove 43, it will be clasped with the second clamping portion 42.

In this embodiment, the second clamping portion 42 is provided at an opening of the annular groove 43, forming a tapered end. During installation, only by inserting the first clamping portion 341 into the annular groove 43, the second clamping portion 42 can be clasped with the first clamping portion 341 securely and is not easy to be separated. Under the action of the elastic force of the flexible part 4 itself and the inclined hook structure, the flexible part 4 is tightened. In this way, a connection between the mounting structure 34 and the flexible part 4 can be tightly connected, thereby achieving a better sealing effect.

This embodiment provides a flexible membrane magnetically driven to generate negative pressure, which serves as the flexible part 4. The flexible membrane includes a second mounting portion 4a and a deformation portion 4b. The second mounting portion 4a is configured for mounting the flexible membrane, and the deformation portion 4b is integrally formed with the second mounting portion 4a. A sealed connection is formed between the deformation portion 4b and the second mounting portion 4a. The second mounting portion 4a is provided at an edge of the deformation portion 4b.

In this embodiment, the deformation portion 4b has a circular plate-like shape. In some embodiments, the deformation portion 4b can be polygonal-shape or other irregular-shapes. The second mounting portion 4a is in an annular-shaped, and is configured to match the shape of the deformation portion 4b. The deformation portion 4b and second mounting portion 4a are integrally formed through injection molding, resulting in the bowl-like shape. The first magnetic part 41 is in a block-shaped, and is provided at a center of the deformation portion 4b. The second mounting portion 4a and the deformation portion 4b are both made of latex. An edge of the second mounting portion 4a is provided with a groove, which is the annular groove 43. The flexible membrane is provided on the mounting structure 34 via the groove structure, allowing for quick engagement of the flexible membrane with the suction bowl, facilitating convenient assembly. The groove is provided on an outer side of the deformation portion 4b, with an opening of the groove facing downward.

A side wall of the annular groove 43 away from the deformation portion 4b is provided with a second clamping portion 42 extending toward the deformation portion 4b. And the second clamping portion 42 is provided at an end of the side wall near an opening of the annular groove 43.

By embedding the first magnetic part 41 within the deformation portion 4b, the deformation portion 4b can achieve the deformation effect by magnetic drive without using the vacuum pump or mechanical mechanism drive. Therefore, the flexible membrane designed herein can effectively prevent the noise generation, and will not affect the user's emotion.

In an embodiment, the third magnetic part 45 is embeddedly provided within the deformation portion 4b, and is provided at a periphery of the first magnetic part 41.

In this embodiment, when the third magnetic part 45 is provided in plurality, a plurality of third magnetic parts 45 are arranged in an ascending manner in diameter from inside to outside along a radial direction of the deformation portion 4b.

In an embodiment, the deformation portion 4b has a wave-shaped cross section.

In this embodiment, the wave-shaped configuration of the deformation portion 4b can enhance its resilience performance. The deformation portion 4b has a plurality of troughs 46, and third magnetic parts 45 are provided at troughs, respectively. In some embodiments, the third magnetic parts 45 has an arc-shaped cross section, allowing it to fit with the troughs 46 of the deformation portion 4b.

This application provides a method for magnetizing a flexible membrane (i.e., the flexible part 4), which is performed as follows.

Firstly, the flexible membrane is integrally formed through an injection molding method.

Secondly, the first magnetic part 41 and the third magnetic part 45 are embeddedly provided within the deformation portion 4b through the following steps. The first magnetic part 41 and the third magnetic part 45 are provided in a suction bowl membrane through a comolding process. At this time, a neodymium alloy in the flexible membrane does not exhibit magnetism. After the comolding process is completed, the flexible membrane (the first magnetic part 41 and the third magnetic part 45 are provided) is placed in a fixture to secure its position and prevent movement. A suction bowl membrane assembly (the first magnetic part 41 and the third magnetic part 45 are provided) is then transferred to a magnetizing apparatus for magnetization to obtain the magnetized flexible membrane provided herein.

The magnetization process is performed through the following steps.
(1) The neodymium alloy (not magnetized) is cleaned to remove the surface oil and dust.
(2) The neodymium alloy surface is treated with a treatment agent for bonding metal to silicone, and dried according to the instructions of the treatment agent to obtain a treated neodymium alloy.
(3) After treatment is completed, the treated neodymium alloy is placed in a mold for comolding to obtain a silicone base assembly.
(4) The silicone base assembly is cleaned to remove the surface oil and dust.
(5) The silicone base assembly is treated with the treatment agent again, and dried according to the instruction of the treatment agent to obtain a treated silicone base assembly.
(6) The treated silicone base assembly is placed in the mold for comolding with the flexible membrane to obtain a semi-finished flexible membrane (neodymium alloy is provided).
(7) The semi-finished flexible membrane (neodymium alloy is provided) is placed into the magnetizing fixture to prevent any movement of the semi-finished suction bowl (neodymium alloy is provided) during the magnetization process, and placed into the magnetizing machine to obtain the magnetized flexible membrane.

In an embodiment, a protrusion 44 is provided on the flexible part 4, more specifically, the protrusion 44 is provided on a side wall of the second mounting portion 4a, close to the deformation portion 4b and away from the annular groove 43.

In this embodiment, after the convex ring 122 is assembled with the suction bowl, the protrusion 44 can also form an interference fit with a side wall of the convex ring 122, enhancing the seal at the connection.

In an embodiment, the first magnetic part 41 and the third magnetic part 45 are both neodymium magnets.

In this embodiment, the neodymium alloy is magnetized to obtain a neodymium magnet.

This application provides a magnetically-driven system, which includes components from the breast pump magnetically driven to generate negative pressure provided herein, including: the flexible part 4, the mounting structure 34, the first magnetic part 41, and the second magnetic part 5. The flexible part 4 is provided on the mounting structure 34. The connection between the flexible part 4 and the mounting structure 34 is sealed. The air hole 47 is provided on the mounting structure 34. The first magnetic part 41 is provided within the flexible part 4. The second magnetic part 5 is provided opposite to the first magnetic part 41, and is configured to alternate between a S pole and a N pole.

In this embodiment, the traditional breast pumping form of vacuum pump and solenoid valve is transformed into the electromagnetic breast pumping form. By alternately switching the second magnetic part 5 between the S pole and N pole, the flexible part 4 is driven to be deformed, generating negative pressure for milk suction. Additionally, given that the vacuum pump and the solenoid valve are not required, noise levels are significantly reduced and may even achieve the “zero” noise. As a result, the magnetically-driven system designed herein can alleviate any impact on the mother's emotions and enhance the overall comfort.

In an embodiment, the magnitude of the magnetic force between the first magnetic part 41 and the second magnetic part 5 (i.e., the electromagnetic coil) can be calculated, expressed as equations (1)-(5):

$$P = \frac{F}{S} ; \qquad ( 1 )$$

$$F = PS ; \qquad ( 2 )$$

where in equations (1) and (2), P is a pressure, F is a force, S is an area;
a conversion between the pressure and the force is 1 Pa=1 N/m², and 1 mmHg=133.3223684 Pa;
thus, by calculating the area of the mounting structure 34, the required magnetic force to pull the flexible part 4 upward can be obtained by substituting the area into the equations (1) and (2);
a Maxwell's electromagnetic attraction is expressed as equation (3):

$$F = \frac{{B}^{2} S}{2 {μ}_{0}} = \frac{{∅}^{2}}{2 {μ}_{0} S} ; \qquad ( 3 )$$

where in equation (3), F is the electromagnetic attraction, B is a magnetic flux density (T), S is a cross-sectional area of magnetic circuit (m²), Ø is a magnetic flux through the cross-section area (Wb), μ₀ is a permeability of vacuum (4π×10⁻⁷ Wb/A·m);
under the assumption that magnetic leakage losses and connection gap losses are negligible, it is considered that the deformation stroke of the mounting structure 34 serves as the primary air gap. At this time, the magnetic flux density (B) at a position of a magnetic component embedded within the mounting structure 34 is expressed as:

$$B = \frac{NU {μ}_{0}}{Rg} = \frac{NI {μ}_{0}}{g} ; \qquad ( 4 )$$

where in equation (4), N is the number of turns in a coil, U is a power supply voltage (V), R is a resistance of a winding coil (Ω), g is an air gap width (m), which is a distance between the magnetic component and the cross-section area of the coil, I is a current strength (A), μ₀ is a permeability of vacuum (4π×10⁻⁷ Wb/A·m);
the equation (4) is substituted into equation (3) to obtain an equation (5), expressed as:

$$F = \frac{{( NI )}^{2} {μ}_{0} S}{2 {g}^{2}} ; \qquad ( 5 )$$

where in equation (5), N is the number of turns in a coil, I is the current strength (A), μ₀ is a permeability of vacuum (4π×10⁻⁷ Wb/A·m), S is the cross-sectional area of magnetic circuit (m²), g is the air gap width (m), which is the distance between the magnetic component and the cross-section area of the coil;
through the above equations (1)-(5), the area of the first magnetic part 41 can be determined, which is the same as the cross-sectional area(S) of the coil magnetic circuit. Additionally, the distance (g) between the first magnetic part 41 and the cross-section of the second magnetic part 5 can also be calculated. The number of turns (N) and the coil current (I) can be adjusted as tuning values, where NI is proportional to F, ensuring that F exceeds the upward pulling force of the flexible part 4, thus meeting the required negative pressure.

This application provides a method for controlling the breast pump magnetically driven to generate negative pressure, which is performed as follows.
(1) The breast shield 11 is provided to form the first cavity, allowing the first cavity to fit snugly and at least partially accommodate the breast to form the sealed cavity.
(2) The flexible part 4 is provided, which includes the first magnetic part 41 configured to at least partially communicate with the sealed cavity and form a portion of the cavity wall of the sealed cavity.
(3) The second magnetic part 5 is provided opposite to the first magnetic part 41, and is configured to drive the first magnetic part 41 to move and drive the flexible part 4 to deform, which causes the sealed cavity to generate negative pressure and facilitate breast milk secretion from mammary glands.

In this embodiment, the traditional breast pumping form of vacuum pump and solenoid valve is transformed into the electromagnetic breast pumping form, significantly reducing the weight of the breast pump for easier portability. Additionally, given that the vacuum pump and the solenoid valve are not required, the second magnetic part 5 is configured to control the movement of the first magnetic part 41, thereby driving the flexible part 4 to deform, generating the negative pressure in the sealed cavity to facilitate breast milk secretion from the mammary glands. This configuration effectively prevents noise generation, noise levels are significantly reduced and may potentially achieve “zero” noise. As a result, the method provided herein can alleviate any impact on the mother's emotions and enhance overall comfort.

In an embodiment, the step (3) further includes the following steps.

The second magnetic part 5 is controlled to alternate between the S pole and the N pole, so that the first magnetic part 41 can drive the flexible part 4 to undergo reciprocating motion.

In an embodiment, the step (1) further includes the following steps.

The breast shield 11 is connected to the connecting part 3 that has the hollow structure. The end of the connecting part 3 is provided with the opening. The tapered end of the breast shield 11 is connected to the opening of the connecting part 3.

The connecting part 3 is provided with the suction bowl. An interior of the suction bowl is communicated with the connecting part 3.

The flexible part 4 is provided within the suction bowl. The place between the breast shield 11 and the flexible part 4 is defined as the sealed cavity through the connecting part 3.

In an embodiment, the step (3) further includes the following steps.

The second magnetic part 5 is fixed within the second housing 2, and is connected to the control mechanism. The control mechanism is provided within the second housing 2.

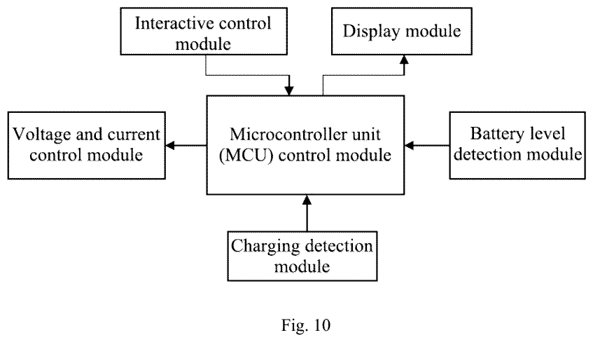

As shown in FIG. 10, this application provides a circuit control system, which is employed in the above control method. The circuit control system includes a microcontroller unit (MCU) control module, an interactive control module, and a voltage and current control module. The MCU control module is configured to receive and emit signals. The interactive control module is configured to receive user's button 211 operation signals and to send these operation signals to the MCU control module. And the voltage and current control module is configured to receive control signals from the MCU control module, control the voltage and current of the electromagnetic coil to generate different suction force, and transmit different suction level signals back to the MCU control module.

In this embodiment, after the user operates the button 211, the interactive control module is configured to send the corresponding button 211 signal to the MCU control module. The MCU control module is configured to receive the signal from the interactive control module and issue a control signal to the voltage and current control module. After receiving the control signal from the MCU control module, the voltage and current control module is configured to control the voltage and current of the electromagnetic coil according to the instructions of the signal, thereby regulating the magnitude of the electromagnetic force, which in turn controls the deformation of the flexible part 4 and ultimately adjusts the generation of desired suction forces.

In an embodiment, the circuit control system further includes a battery level detection module, which is configured to monitor the battery level of the power supply device 7 in real time and send the data information to the MCU control module.

In this embodiment, the battery level detection module is configured to monitor the power level of the internal battery of the breast pump in real time and transmit the monitoring data to the MCU control module.

In an embodiment, the circuit control system further includes a charging detection module, which is configured to detect the external charger and provide feedback to the MCU control module.

In this embodiment, when the battery of the breast pump is depleted, the charging detection module will detect the charger and monitor the charging status in real time when charging is initiated.

In an embodiment, the circuit control system further includes a display module, which is configured to receive signals from the MCU control module, where these signals are sent from the voltage and current control module, the battery level detection module, and the charging detection module.

In this embodiment, after the voltage and current control module, the battery level detection module, and the charging detection module send signals to the MCU control module, the MCU control module will transmit these signals to the display module, which will display the data information on the screen.

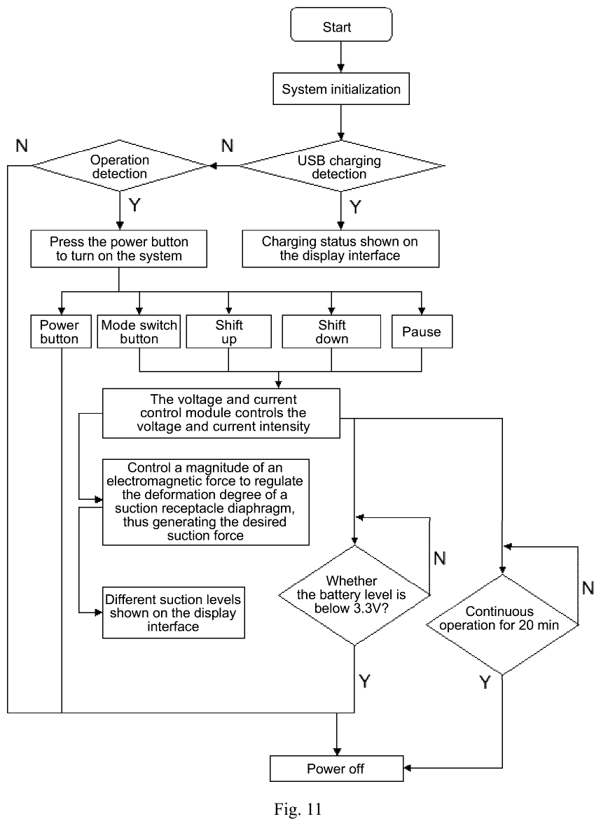

As shown in FIG. 11, this application provides a circuit control method to control the above circuit control system, which is performed as follows.
(1) The power button is pressed to turn on and initialize the system.
(2) Whether an external charger is connected is determined by the charging detection module. If there is an external charger connected, the charging status signal is transmitted to the display module and displayed.
(3) Whether there is an operation instruction for the button 211 is determined by the interactive control module. If there is an operation instruction for the button 211, the instruction is sent to the MCU control module; if not, the system will be automatically shut down.
(4) The instruction sent by the interactive control module is received, judged and transmitted to the voltage and current control module by the MCU control module.
(5) After receiving the instruction, the voltage and current control module controls the voltage and current intensities supplied to the electromagnetic coil, thereby regulating the magnitude of the electromagnetic force to generate the desired suction force.

In this embodiment, before performing any operation, it is necessary to first initialize the system of the breast pump. After initialization, the user operates the button 211. Once the interactive control module determines that there is the operation instruction for the button 211, the power button is pressed to turn on the system. This setup prevents the breast pump from accidentally starting caused by unintended operation. Once the system is on, the button 211 can be used for control. The interactive control module sends the corresponding instruction of button 211 to the MCU control module, which then sends this instruction to the voltage and current control module. The voltage and current control module adjusts the voltage and current intensity supplied to the electromagnetic coil based on the instruction, thereby regulating the magnitude of the electromagnetic force, which in turn controls the deformation of the flexible part 4 to generate different suction force. The different suction levels are then fed back to the display module for display.

After turning on the system, if the power button is pressed again, the breast pump will shut down.

In an embodiment, the button 211 operation instructions include mode switch button instruction, shift up instruction, shift down instruction, and pause instruction.

In this embodiment, the mode switch button instruction, shift up instruction, shift down instruction, and pause instruction correspond to the mode switch button, the “+” button for shift up, the “−” button for shift down, and the pause button on the breast pump. Pressing the corresponding button will issue the respective instruction.

In an embodiment, after the breast pump starts working, the circuit control method further includes the battery level detection module determining whether the voltage is below the minimum operating voltage. If it is below the minimum operating voltage, the breast pump will automatically shut down; if it is above the minimum operating voltage, the breast pump will continue to operate.

In this embodiment, the minimum operating voltage is 3.3V. If the voltage is below 3.3V, the breast pump will automatically shut down; if it is above 3.3V, the breast pump will continue to operate.

In an embodiment, after the breast pump starts working, the circuit control method further includes the MCU control module determining whether the preset continuous operation duration has been reached. If it has been reached, the breast pump will automatically shut down. If not, the breast pump will continue to operate.

In this embodiment, the preset continuous operation duration is 20 min. If the continuous operation reaches 20 min, the breast pump will automatically shut down. If it is less than 20 min, the breast pump will continue to operate.

Described above are merely preferred embodiments of the present disclosure, and are not intended to limit the scope of the present disclosure. It should be understood that various modifications, changes and replacements made by those skilled in the art without departing from the spirit of the disclosure shall fall within the scope of the present disclosure defined by the appended claims.

What is claimed is:

1. A flexible membrane magnetically driven to generate negative pressure, comprising:
a mounting portion;
a deformation portion; and
a first magnetic part;
wherein the mounting portion is configured for mounting the flexible membrane;
the deformation portion is integrally formed with the mounting portion, a connection between the deformation portion and the mounting portion is sealed, and the mounting portion is provided at an edge of the deformation portion; and
the first magnetic part is embeddedly provided within the deformation portion.

2. The flexible membrane of claim 1, wherein a groove is provided at an edge of the mounting portion.

3. The flexible membrane of claim 1, further comprising:
at least one second magnetic part;
wherein the at least one second magnetic part is embeddedly provided within the deformation portion, and is provided at a periphery of the first magnetic part.

4. The flexible membrane of claim 3, wherein the at least one second magnetic part is configured to surround the first magnetic part.

5. The flexible membrane of claim 3, wherein the deformation portion has a wave-shaped cross section.

6. The flexible membrane of claim 5, wherein the at least one second magnetic part is provided in plurality; each of a plurality of second magnetic parts is in an annular shape; and the plurality of second magnetic parts are provided spaced apart in the deformation portion;
the plurality of second magnetic parts are arranged in an ascending manner in diameter from inside to outside along a radial direction of the flexible membrane; and
the deformation portion has a plurality of troughs, and the plurality of second magnetic parts are provided at the plurality of troughs, respectively.

7. The flexible membrane of claim 2, wherein the groove is configured as an annular groove;
a side wall of the annular groove away from the deformation portion is provided with a clamping portion extending toward the deformation portion; and the clamping portion is provided at an end of the side wall near an opening of the annular groove; and
the clamping portion is configured to form a hook-shaped structure with the side wall.

8. The flexible membrane of claim 7, wherein a protrusion is provided on a side wall of the mounting portion close to the deformation portion and away from the annular groove.

9. The flexible membrane of claim 3, wherein the first magnetic part and the at least one second magnetic part are each a neodymium magnet.

10. A magnetically-driven breast pump, comprising:
the flexible membrane of claim 1.
